(19) United States
(12) Patent Application Publication
(10) Pub. No.: US 2004/0213828 A1
(43) Pub. Date: Oct. 28, 2004

(54) Pain relief lollipop compositions and methods

(76) Inventor: David J. Smith, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/422,931

(22) Filed: Apr. 23, 2003

(51) Int. Cl. A61K 31/66; A61K 31/5513; A61K 31/522; A61K 9/68

(52) U.S. Cl. 424/440; 514/221; 514/263.31; 514/649; 514/114

(57) ABSTRACT

A pain relief lollipop comprises a candy matrix comprising (a) an opioid agonist, (b) an N-methyl-D-aspartate receptor antagonist different from the opioid agonist, and, optionally, a muscle relaxant, sedative, anxiolytic, and/or antidepressant. A patient can self-administer small amounts of the pain relief drug as needed by simply licking or sucking on the lollipop in response to his subjective experience of pain.

### BACKGROUND OF THE INVENTION

1. Field of the Invention

This invention relates to compositions useful for alleviating pain and systems for their delivery to humans. More particularly, this invention relates to pain relief lollipops that may be used to provide oral transmucosal delivery of opioid agonists and NMDA receptor antagonists.

2. Description of the Related Art

The treatment of physical pain concerns health care professionals throughout the world. The treatment of chronic pain is particularly challenging because of the frequent need for repeated administration of pain relief medication. Chronic pain is generally considered to be pain that continues a month or more beyond the usual recovery period for an illness or injury or pain that goes on over months or years as a result of a chronic condition. It may be continuous or come and go. It is estimated that chronic pain disables, to some degree, about 86 million Americans. It is regarded as a source of frustration for the health care professionals who care for the patient, and affects the quality of life and economic security not only of the person with pain, but also his or her family. It is estimated that United States business and industry loses about $90 billion annually to sick time, reduced productivity, and direct medical and other benefit costs due to chronic pain among employees. In some cases, repeated administration of the pain relief medication causes sufferers of chronic pain to develop an undesirable tolerance or addiction, creating further health issues for the patient and additional challenges for the health care professional.

There are a number of methods for administering pain relief medications, including oral and parenteral (administered in a manner other than through the digestive tract). Oral administration is most frequently accomplished by formulating the pain relief medication into tablet or syrup and allowing the patient to swallow it. This method is simple, well accepted and relatively painless, but may be problematic for uncooperative patients. Also, there is often a considerable lapse of time between administration of the pain relief medication and its therapeutic effect because of the time needed for gastrointestinal absorption. This time lag is of particular concern when a patient is suffering from severe or chronic pain. Faster administration may be accomplished by direct injection of the pain relief medication, but most people consider the injection itself to be painful and thus undesirable. What is needed is a method for administering a variety of pain relief formulations that is fast, well tolerated and relatively painless.

### SUMMARY OF THE INVENTION

The invention is directed towards pain relief delivery systems and methods and, in particular, lollipops and methods of using them for the relief of pain. Preferred lollipops are capable of introducing a pain relief drug into a patient's system faster than a pain relief drug taken orally and without the pain and invasiveness of an injection. Unlike the ordinary oral ingestion of pain medication or injections where a relatively large dose is given intermittently, use of a pain relief lollipop may permit the patient to take relatively small doses of pain relief medication on an almost continuous basis. A patient may self-administer small amounts of the pain relief drug as needed by simply licking or sucking on the lollipop in response to his or her subjective experience of pain. These significant advantages can be achieved by incorporating pain relief drugs into a candy mixture capable of being absorbed by the patient through the mucosal tissue of the mouth, pharynx and esophagus. The candy and pain relief drug is molded into a lollipop form, which, as discussed more specifically hereinafter, may be administered by the patient as needed for pain relief.

Preferred pain relief drugs are opioid agonists. To inhibit the development of tolerance and/or addiction to the opioid agonists by the patient, preferred lollipops further comprise a substance that blocks the N-methyl-D-aspartate receptor, herein referred to as an “NMDA receptor antagonist.” Preferred lollipops also contain a muscle relaxant, a sedative, an anxiolytic, and/or tricyclic antidepressant, to provide further comfort and relief for the patient.

A preferred embodiment provides a pain relief lollipop, comprising a candy matrix comprising (a) an opioid agonist, (b) an N-methyl-D-aspartate receptor antagonist different from the opioid agonist, and (c) a medicament selected from the group consisting of a muscle relaxant, a sedative, an anxiolytic and a tricyclic antidepressant, the medicament being different from the opioid agonist and different from the N-methyl-D-aspartate receptor antagonist; and a holder in contact with the candy matrix. Another preferred embodiment provides a method for treating pain, comprising identifying a human suffering from pain and administering the pain relief lollipop to the human.

Another preferred embodiment provides a candy matrix comprising (a) an opioid agonist selected from the group consisting of fentanyl, hydromorphone, hydrocodone, oxycodone, oxymorphone, propoxyphene, sulfentanil, and pharmaceutically acceptable salts thereof, and (b) an N-methyl-D-aspartate receptor antagonist selected from the group consisting of amantadine, dextromethorphan, and pharmaceutically acceptable salts thereof; and a holder in contact with the candy matrix. Another preferred embodiment provides a method for treating pain, comprising identifying a human suffering from pain and administering the pain relief lollipop to the human.

These and other embodiments are described in greater detail below.

### DETAILED DESCRIPTION OF THE PREFERRED EMBODIMENTS

Preferred compositions provide lollipop compositions and methods for using them to treat pain. In this context, “lollipop” is used in its ordinary sense to refer to a piece of candy in operable contact with a holder. Preferably, the candy is supported by, held by, or attached to the holder to provide operable contact. Non-limiting examples of holders include a stick or string partially embedded in or attached to the candy, and a platform such as a cone that supports the candy.

To provide pain relief to the consumer of the lollipop, the candy preferably comprises an opioid agonist. In this context, “opioid agonist” is used in the ordinary sense to refer to opiates, opiate derivatives, opioids, and other substances whose effects are mediated by the same receptor, and includes mixtures thereof. Non-limiting examples of preferred opioid agonists include fentanyl, hydromorphone, hydrocodone, ketamine, methadone, oxycodone, oxymorphone, propoxyphene, sulfentanil, and pharmaceutically acceptable salts thereof.

The amount of opioid agonist in the lollipop is an amount that is effective to reduce the patient's pain, and will generally vary depending on the type and amount of pain experienced by the patient, the characteristics of the patient, and the efficacy of the particular opioid agonist. Dosages of particular opioid agonists useful for treating pain are known to those skilled in the art, see, e.g., Physician's Desk Reference 2003, which is hereby incorporated by reference in its entirety and particularly for the purpose of describing typical dosages of opioid agonists, NMDA receptor antagonists, muscle relaxants, sedatives, anxiolytics and antidepressants. Dosages may also be determined by routine experimentation. Such known or determined dosages of opioid agonist may be used as a guideline for determining the amount of opioid agonist to include in the lollipop, typically taking into account various patient characteristics (e.g., level of pain, age, weight, and overall health) in a manner known to those skilled in the medical arts, as well as the characteristics of the lollipop (e.g., rate of dissolution) and the transmucosal delivery characteristics of the particular opioid agonist.

To avoid or reduce the development of tolerance and/or dependence by the patient on the opioid agonist, the lollipop candy preferably comprises an NMDA receptor antagonist different from the opioid agonist. NMDA receptor antagonists are substances known to those skilled in the art that block the N-methyl-D-aspartate receptor or that block a major intracellular consequence of NMDA receptor activation, see U.S. Pat. Nos. 5,321,012; 5,654,281 and 5,869,498, all of which are hereby incorporated by reference in their entireties, and particularly for the purpose of describing NMDA receptor antagonists and their uses. The NMDA receptor antagonist may be a mixture. Non-limiting examples of preferred NMDA receptor antagonists for inclusion in the lollipop include amantadine, D,L-2-amino-5-phosphono valeric acid, dextromethorphan, ketamine, methadone, and pharmaceutically acceptable salts thereof. Since some substances, e.g., ketamine and methadone, may be classified as both opioid agonists and NMDA receptor antagonists, it is understood that the opioid agonist in any particular lollipop is different from the NMDA receptor antagonist.

The amount of NMDA receptor antagonist in the lollipop is an amount that is effective to avoid or reduce the development of tolerance and/or dependence by the patient on the opioid agonist, and will generally vary depending on the type and amount of the particular opioid agonist present in the lollipop and on the effectiveness of the particular NMDA receptor antagonist. Dosages of particular NMDA receptor antagonists useful for avoiding or reducing the development of tolerance and/or dependence to a particular opioid agonist are known to those skilled in the art, see, e.g., Physician's Desk Reference 2003, or may be determined by routine experimentation. Such known or determined dosages of NMDA receptor antagonist may be used as a guideline for determining the amount of the NMDA receptor antagonist to include in the lollipop, typically taking into account various patient characteristics (e.g., level of pain, age, weight, and overall health) in a manner known to those skilled in the medical arts, as well as the characteristics of the lollipop (e.g., rate of dissolution) and the transmucosal delivery characteristics of the particular NMDA receptor antagonist.

For example, a preferred pain relief lollipop comprises a candy matrix comprising (a) an opioid agonist selected from the group consisting of fentanyl, hydromorphone, hydrocodone, oxycodone, oxymorphone, propoxyphene, sulfentanil, and pharmaceutically acceptable salts thereof; and (b) an N-methyl-D-aspartate receptor antagonist selected from the group consisting of amantadine, dextromethorphan, and pharmaceutically acceptable salts thereof; and a holder in contact with the candy matrix.

Pain relief lollipops may optionally contain additional medicaments to provide further comfort and/or relief to the patient, and/or to treat specific conditions such a muscle tension, overexcitement, anxiety and/or depression. Preferably, the lollipop candy further comprises a medicament selected from the group consisting of a muscle relaxant, a sedative, an anxiolytic and an antidepressant, the medicament being different from the opioid agonist and different from the N-methyl-D-aspartate receptor antagonist. The terms “muscle relaxant,” “sedative,” “anxiolytic” and “antidepressant” are known to those skilled in the art, see, e.g., Physician's Desk Reference 2003 and Dorland's Illustrated Medical Dictionary, W.B. Saunders Co., 2000, and include mixtures thereof Non-limiting examples of preferred muscle relaxants include alprazolam, butalbital, clonazepam, carisoprodol, diazepam, flexeril, lorazepam, methocarbamol, and pharmaceutically acceptable salts thereof. Non-limiting examples of sedatives include clonazepam, butalbital, diazepam, phenobarbital, and pharmaceutically acceptable salts thereof. Non-limiting examples of anxiolytics include alprazolam, clonazepam, diazepam, lorazepam, and pharmaceutically acceptable salts thereof. Non-limiting examples of antidepressants include tricyclic antidepressants and monamine oxidase inhibitors. Non-limiting examples of preferred tricyclic antidepressants include amitriptyline, amoxapine, desipramine, doxepin, imipramine, maprotiline, nortriptyline, protriptyline, trimipramine, and pharmaceutically acceptable salts thereof. Those skilled in the art recognize that some substances may have multiple classifications (e.g., alprazolam as both a muscle relaxant and an anxiolytic). It is therefore understood that reference herein to a lollipop that contains two or more medicaments selected from the group consisting of opioid agonist, NMDA receptor antagonist, muscle relaxant, sedative, anxiolytic and antidepressant, means that each of the two or more medicaments are different from one another.

The amount and type of additional medicament (e.g., muscle relaxant, sedative, anxiolytic and/or antidepressant) in the lollipop, if any, is an amount that is effective to provide additional comfort and/or relief to the patient from various conditions, and will generally vary depending on the type and amount of the particular additional medicament present in the lollipop. Dosages of particular additional medicaments useful for treating various conditions are known to those skilled in the art, see, e.g., Physician's Desk Reference 2003, or may be determined by routine experimentation. Such known or determined dosages of additional medicaments may be used as a guideline for determining the amount of the additional medicament(s) to include in the lollipop, typically taking into account various patient characteristics (e.g., level of muscle tension, overexcitement, anxiety and/or depression, age, weight, and overall health) in a manner known to those skilled in the medical arts, as well as the characteristics of the lollipop (e.g., rate of dissolution) and the transmucosal delivery characteristics of the particular medicament(s).

Pain relief lollipops are preferably made by intermixing the medicaments (e.g., opioid agonist, NMDA receptor antagonist, and, optionally, muscle relaxant, sedative, anxiolytic and/or antidepressant) with the other non-medicinal lollipop ingredients (e.g., candy, sorbitol, flavoring, coloring, etc.) during the making of the lollipops. The non-medicinal lollipop ingredients are preferably selected to slowly dissolve and/or melt in the patient's mouth in a manner similar to that of a traditional lollipop, so as to release the medicaments over the course of dissolution and/or melting. The medicaments may be incorporated into the lollipop in other ways, e.g. by coating onto the exterior of a lollipop, but incorporation during manufacture is generally preferable so that the medicaments are more uniformly dispersed throughout the resulting candy matrix. Such relatively uniform dispersal has been found to provide a corresponding uniform release and subsequent transmucosal absorption of the medicaments during administration of the lollipop to the patient.

Methods for making conventional lollipops are well known in the art. A preferred embodiment of the invention provides a method for making a pain relief lollipop, comprising intermixing the various medicaments and non-medicinal ingredients in the molten or partially molten state by heating the ingredients to a temperature effective to melt or partially melt at least part of the ingredients, preferably at a temperature in the range of about 35° C. to about 95° C., pouring the resulting molten or partially molten lollipop mixture into a mold or molds, placing a stick, string or other suitable object into the molded mixture so that part protrudes to serve as a holder, and cooling the lollipop mixture to form a hardened or semi-hardened candy attached to the holder. In an alternative embodiment, the holder is placed in contact with, e.g., attached to, the hardened or semi-hardened candy after cooling. Various methods for making the lollipop may be practiced according to the knowledge of those skilled in the art, although extreme manufacturing conditions that significantly alter the efficacy of the medicaments are to be avoided.

A preferred embodiment provides methods for treating pain, comprising identifying a human suffering from pain and administering a pain relief lollipop as described herein to the human. Methods for identifying humans suffering from pain and assessing the appropriate method of providing relief are known to those skilled in the medical arts. Administration of the pain relief lollipop is preferably by patient self-administration, e.g., by sucking or licking the lollipop, such that oral transmucosal delivery of the various medicaments is facilitated. The pain relief lollipop may be chewed and swallowed to provide gastrointestinal delivery of the medicaments, but administration of the lollipop is preferably conducted by advising the patient not to chew or swallow pieces of the lollipop in order to provide preferred oral transmucosal delivery. Other preferred embodiments comprise identifying a patient suffering from pain as well as muscle tension, overexcitement, anxiety and/or depression, and administering a pain relief lollipop further comprising a muscle relaxant, sedative, anxiolytic and/or antidepressant, to treat such muscle tension, overexcitement, anxiety and/or depression, respectively.

The pain relief lollipops described herein are particularly useful for the treatment of patients suffering from chronic pain because such treatment may involve multiple administrations over an extended period of time. In such situations, the preferred pain relief lollipops described herein may be used to provide numerous benefits, including relief of the primary pain by the opioid agonist and avoidance or reduction of the development of tolerance and/or dependence by the patient to the opioid agonist, as well as treatment of other conditions which may be present such as muscle tension, overexcitement, anxiety and/or depression, by the administration of additional medicaments such as muscle relaxants, sedatives, anxiolytics and antidepressants, respectively.

### EXAMPLE 1

Eighteen suppository mold strips (each containing 12 molds) in a metal-holding tray were set up on top of wax paper in a vertical flow hood. The mold strips were sprayed with cooking oil spray and a rubber spatula was used across the tops of the molds to push oil inside. A spin bar was placed into a 500 milliliters (mL) beaker and about 529 grams (g) of a commercially available sorbitol lollipop base was added to the beaker. The contents were heated on a hot plate at about 70° C. with magnetic stirring to melt the lollipop base. About 0.9 g of hydromorphone was weighed in the barrel of a 20 mL syringe. Water and red food coloring were drawn into the syringe and the resulting mixture was shaken to provide a hydromorphone solution. The hydromorphone solution was then injected into the melted lollipop base over the course of several minutes with stirring. A uniform red color indicated complete dispersion of the ingredients. The temperature of the molten mixture was reduced to about 50° C. A mixture of 0.45 g dextromethorphan, 0.3 g silica gel, and 1.1 g sodium saccharin were intermixed and added to the mixture in the beaker with stirring. About 5 mL raspberry flavor and about 3 mL wild cherry oil were then added with stirring to the mixture in the beaker to form a lollipop solution. The lollipop solution was then drawn into a 60 mL syringe, which was then used to fill each of the 216 suppository molds. One end of a 2-3 inch plastic stick was then placed near the center of each lollipop to form a handle. The lollipops in the mold were then refrigerated overnight, then removed from the molds. Each of the resulting lollipops contained about 4 milligrams (mg) of hydromorphone and about 2 mg of dextromethorphan.

### EXAMPLE 2

Eighteen suppository mold strips (each containing 12 molds) in a metal-holding tray were set up on top of wax paper in a vertical flow hood. The mold strips were sprayed with cooking oil spray and a rubber spatula was used across the tops of the molds to push oil inside. A spin bar was placed into a 500 mL beaker and about 535 g of a commercially available sorbitol lollipop base was added to the beaker. The contents were heated on a hot plate at about 90° C. with magnetic stirring to melt the lollipop base. About 0.42 g of fentanyl citrate was weighed in the barrel of a 20 mL syringe. Water and red food coloring were drawn into the syringe and the resulting mixture was shaken to provide a hydromorphone solution. The hydromorphone solution was then injected into the melted lollipop base over the course of several minutes with stirring. A uniform red color indicated complete dispersion of the ingredients. The temperature of the molten mixture was reduced to about 50° C. A mixture of 0.45 g dextromethorphan, 0.3 g silica gel, and 1.1 g sodium saccharin were intermixed and added to the mixture in the beaker with stirring. About 5 mL raspberry flavor and about 3 mL wild cherry oil were then added with stirring to the mixture in the beaker to form a lollipop solution. The lollipop solution was then drawn into a 60 mL syringe, which was then used to fill each of the 216 suppository molds. One end of a 2-3 inch plastic stick was then placed near the center of each lollipop to form a handle. The lollipops in the mold were then refrigerated overnight, then removed from the molds. Each of the resulting lollipops contained about 1.25 mg of fentanyl and about 2 mg of dextromethorphan.

### EXAMPLE 3

The procedure described in Example 2 was repeated except that a smaller amount (0.36 g) of fentanyl citrate was used. Each of the resulting lollipops contained about one mg of fentanyl and about 2 mg of dextromethorphan.

### EXAMPLE 4

The procedure described in Example 2 was repeated except that a smaller amount (0.18 g) of fentanyl citrate was used. Each of the resulting lollipops contained about 500 micrograms of fentanyl and about 2 mg of dextromethorphan.

### EXAMPLE 5

The procedure described in Example 2 is repeated except that 2.16 g of methocarbamol is added to the mixture of 0.45 g dextromethorphan, 0.3 g silica gel, and 1.1 g sodium saccharin. Each of the resulting lollipops contains about 1.25 mg of fentanyl, 10 mg methocarbamol and about 2 mg of dextromethorphan.

Although the foregoing invention has been described in terms of certain preferred embodiments, other embodiments will become apparent to those of ordinary skill in the art in view of the disclosure herein. Accordingly, the invention is not intended to be limited by the recitation of preferred embodiments, but is intended to be defined solely by reference to the appended claims.

What is claimed is:

1. A pain relief lollipop, comprising
a candy matrix comprising (a) an opioid agonist, (b) an N-methyl-D-aspartate receptor antagonist different from the opioid agonist, and (c) a medicament selected from the group consisting of a muscle relaxant, a sedative, an anxiolytic and an antidepressant, the medicament being different from the opioid agonist and different from the N-methyl-D-aspartate receptor antagonist; and
a holder in contact with the candy matrix.

2. The pain relief lollipop of claim 1 in which the opioid agonist is selected from the group consisting of fentanyl, hydromorphone, hydrocodone, ketamine, methadone, oxycodone, oxymorphone, propoxyphene, sulfentanil, and pharmaceutically acceptable salts thereof.

3. The pain relief lollipop of claim 1 in which the N-methyl-D-aspartate receptor antagonist is selected from the group consisting of amantadine, D,L-2-amino-5-phosphono valeric acid, dextromethorphan, ketamine, methadone, and pharmaceutically acceptable salts thereof.

4. The pain relief lollipop of claim 1 in which the medicament is selected from the group consisting of alprazolam, butalbital, carisoprodol, clonazepam, diazepam, flexeril, lorazepam, methocarbamol, phenobarbital, amitriptyline, amoxapine, desipramine, doxepin, imipramine, maprotiline, nortriptyline, protriptyline, trimipramine, and pharmaceutically acceptable salts thereof.

5. The pain relief lollipop of claim 1 in which the medicament is a muscle relaxant selected from the group consisting of alprazolam, butalbital, carisoprodol, clonazepam, diazepam, flexeril, lorazepam, methocarbamol, and pharmaceutically acceptable salts thereof.

6. The pain relief lollipop of claim 1 in which the medicament is a sedative selected from the group consisting of clonazepam, butalbital, diazepam, phenobarbital, and pharmaceutically acceptable salts thereof.

7. The pain relief lollipop of claim 1 in which the medicament is a tricyclic antidepressant selected from the group consisting of amitriptyline, amoxapine, desipramine, doxepin, imipramine, maprotiline, nortriptyline, protriptyline, trimipramine, and pharmaceutically acceptable salts thereof.

8. The pain relief lollipop of claim 1 in which the medicament is an anxiolytic selected from the group consisting of alprazolam, clonazepam, diazepam, lorazepam, and pharmaceutically acceptable salts thereof.

9. A method for treating pain, comprising identifying a human suffering from pain and administering the pain relief lollipop of claim 1 to the human.

10. The method of claim 9, in which the pain is chronic pain.

11. A method for making the pain relief lollipop of claim 1, comprising intermixing the opioid agonist, N-methyl-D-aspartate receptor antagonist, medicament and candy matrix at a temperature effective to at least partially melt the candy matrix to form an at least partially molten lollipop mixture, molding the at least partially molten lollipop mixture to form a molded lollipop mixture, contacting the molded lollipop mixture with a holder, and cooling the molded lollipop mixture to form a pain relief lollipop.

12. A method for making the pain relief lollipop of claim 1, comprising intermixing the opioid agonist, N-methyl-D-aspartate receptor antagonist, medicament and candy matrix at a temperature effective to at least partially melt the candy matrix to form an at least partially molten lollipop mixture, molding the at least partially molten lollipop mixture to form a molded lollipop mixture, cooling the molded lollipop mixture to form an at least partially hardened candy, and contacting the at least partially hardened candy with a holder to form a pain relief lollipop.

13. A pain relief lollipop, comprising
a candy matrix comprising (a) an opioid agonist, (b) an N-methyl-D-aspartate receptor antagonist different from the opioid agonist, and (c) a medicament selected from the group consisting of a muscle relaxant, a sedative, an anxiolytic and an antidepressant, the medicament being different from the opioid agonist and different from the N-methyl-D-aspartate receptor antagonist; and
a means for holding the candy matrix.

14. A pain relief lollipop, comprising
a candy matrix comprising (a) an opioid agonist selected from the group consisting of fentanyl, hydromorphone, hydrocodone, oxycodone, oxymorphone, propoxyphene, sulfentanil, and pharmaceutically acceptable salts thereof, and (b) an N-methyl-D-aspartate receptor antagonist selected from the group consisting of amantadine, dextromethorphan, and pharmaceutically acceptable salts thereof; and
a holder in contact with the candy matrix.

15. The pain relief lollipop of claim 14 in which the candy matrix further comprises a muscle relaxant.

16. The pain relief lollipop of claim 15 in which the muscle relaxant is selected from the group consisting of alprazolam, butalbital, clonazepam, carisoprodol, diazepam, flexeril, lorazepam, methocarbamol, and pharmaceutically acceptable salts thereof.

17. The pain relief lollipop of claim 14 in which the candy matrix further comprises a sedative.

18. The pain relief lollipop of claim 17 in which the sedative selected from the group consisting of clonazepam, butalbital, diazepam, phenobarbital, and pharmaceutically acceptable salts thereof.

19. The pain relief lollipop of claim 14 in which the candy matrix further comprises an anxiolytic.

20. The pain relief lollipop of claim 19 in which the anxiolytic is selected from the group consisting of alprazolam, clonazepam, diazepam, lorazepam, and pharmaceutically acceptable salts thereof.

21. The pain relief lollipop of claim 14 in which the candy matrix further comprises an antidepressant.

22. The pain relief lollipop of claim 21 in which the antidepressant is a tricyclic antidepressant is selected from the group consisting of amitriptyline, amoxapine, desipramine, doxepin, imipramine, maprotiline, nortriptyline, protriptyline, trimipramine, and pharmaceutically acceptable salts thereof.

23. The pain relief lollipop of claim 14 in which the candy matrix further comprises at least two different medicaments selected from the group consisting of a muscle relaxant, a sedative, an anxiolytic and a tricyclic antidepressant.

24. The pain relief lollipop of claim 23 in which the muscle relaxant is selected from the group consisting of alprazolam, butalbital, clonazepam, diazepam, flexeril, lorazepam, methocarbamol, and pharmaceutically acceptable salts thereof; in which the sedative is selected from the group consisting of clonazepam, butalbital, diazepam, phenobarbital, and pharmaceutically acceptable salts thereof; in which the anxiolytic is selected from the group consisting of alprazolam, clonazepam, diazepam, lorazepam, and pharmaceutically acceptable salts thereof; and in which the tricyclic antidepressant is selected from the group consisting of amitriptyline, amoxapine, desipramine, doxepin, imipramine, maprotiline, nortriptyline, protriptyline, trimipramine, and pharmaceutically acceptable salts thereof.

25. A method for treating pain, comprising identifying a human suffering from pain and administering the pain relief lollipop of claim 14 to the human.

26. The method of claim 25, in which the pain is chronic pain.
